(19) United States
(12) Patent Application Publication
(10) Pub. No.: US 2013/0116575 A1
(43) Pub. Date: May 9, 2013

(54) IMPLANTABLE DOPPLER BLOOD FLOW MONITOR AND DOPPLER PROBE

(71) Applicants: Marlin MICKLE, Pittsburgh, PA (US); Michael A. ROTHFUSS, Feasterville, PA (US); Michael L. GIMBEL, Pittsburgh, PA (US)

(72) Inventors: Marlin MICKLE, Pittsburgh, PA (US); Michael A. ROTHFUSS, Feasterville, PA (US); Michael L. GIMBEL, Pittsburgh, PA (US)

(21) Appl. No.: 13/668,656

(22) Filed: Nov. 5, 2012

Related U.S. Application Data
(60) Provisional application No. 61/555,642, filed on Nov. 4, 2011.

(51) Int. Cl. A61B 5/0265

(52) U.S. Cl. 600/479

(57) ABSTRACT

A Doppler blood flow monitoring system comprises a sensor cuff capable of being secured around an exterior surface of a patient's vessel, at least one transducer attached to the sensor cuff for generating signals into the vessel and for receiving said signals, and a transmitter in communication with the at least one transducer and capable of wirelessly transmitting an RF signal external to the patient's body. The system also comprises a receiver capable of receiving the RF signal from the transmitter and being capable of generating an audible sound or signal commensurate with detected flow. The system can take advantage of pulse wave or continuous wave Doppler technology.

### CROSS-REFERENCE TO RELATED APPLICATION

This application is based upon and claims the priority of U.S. Provisional Patent Application Ser. No. 61/555,642, filed Nov. 4, 2011, which is incorporated herein by reference in its entirety

### FIELD OF THE INVENTION

This invention relates to a device for detecting blood flow. More particularly, this invention relates to a method and device for detecting blood flow using an implantable wireless Doppler blood flow monitor.

### BACKGROUND OF THE INVENTION

Free tissue transfer is the removal of tissue from one part of the body to attach it to another region of the body. More particularly, free tissue transfer comprises vascular, i.e., arterial or venous, detachment and then reattachment of these same vessels at a transfer site. An anastomosis, in the tree tissue context, is the connection of two vessels, and anastomoses can be classified into arterial anastomoses and venous anastomoses. An anastomosis facilitates perfusion of blood to the transferred tissue.

Microvascular anastomotic failures are highly problematic. Anastomotic failures require immediate surgical attention, as their failure equates to total or partial loss of blood supply to the transferred tissue. The anastomotic surgery itself causes a change in the clotting nature of blood, inducing a greatly increased capacity for coagulation and thereby thrombosis. The presence of sutures at the anastomotic site can lead to occlusion and thus thrombosis. Kinking or compression of a vessel leads to decreased blood flow and blood stasis. Blood stasis has the dreaded complication of triggering both local coagulation at the anastomotic site and coagulation upstream or downstream in the microcirculatory system. Failure to correct the problem often results in necrotic tissue and, in the case of digit or hand reattachment, amputation of an appendage.

In various types of surgery, including heart, long, and liver transplants, it is necessary to monitor blood flow to insure proper circulation. The patient is tethered to the equipment, and the patient's hospital stay is often unduly prolonged, incurring higher healthcare costs. One method of monitoring blood flow is to use a Doppler blood flow ultrasound monitor where a collar is wrapped around or adjacent to the junction of two vessel ends and a small sensor element that is apart of the collar is connected by fine wires to a benchtop or bedside monitor. The flow monitor detects the frequency shift observed by the sensor element relative to a transmitted wave through flowing blood. An example of such a device is commercially available as the Cook-Swartz Doppler Flow Monitoring System (Cook Vascular, Inc., Vandergrift Pa.). An audible response of the blood flowing through the vessel can be heard. The implanted probe (sensor) is connected to a bedside monitor by wires; however, these wires can be problematic due to inadvertent premature wire removal and the potential to disrupt the blood vessels during purposeful wire removal.

Wireless implantable medical devices are electronic devices that are implanted beneath the surface (possibly deeply into tissue) of a patient's skin. The most well known wireless implantable medical devices are the pacemakers and defibrillators that have been used tor decades. Many of these devices are designed to wirelessly communicate with external monitors, which facilitates popular telemedicine initiatives.

Wireless implantable medical devices have seen incredible growth in complexity and capacity since the first days of pacemakers and defibrillators. Today's devices are designed using custom integrated circuits with thousands and even millions of transistors to provide more functionality and better performance while requiring less power in a smaller footprint. Primarily, these implanted devices are limited by their power supply, which is most often a battery. The power supply limits all aspects of die implanted system including lifetime, maximum communication bandwidth, and capacity to integrate sensors. There is clearly a need for an implantable Doppler blood flow monitoring system that wirelessly transmits signals to remote devices.

### OBJECTS OF THE INVENTION

It is an object of this invention to provide a wireless implantable Doppler blood flow monitor for measuring blood flow in a patient.

It is also an object of this invention to provide an ultra-small, ultra-low power, implantable Doppler blood flow monitor for measuring blood flow in a vessel.

It is a further object of the invention to provide an implantable continuous wave Doppler blood flow monitor that avoids problems with transcutaneous wires.

It is yet a further object of the invention to provide an implantable Doppler blood flow monitor where components can be easily removed from the patient.

It is a yet further object of the invention to provide a method of monitoring blood flow in a patient where signals are wirelessly transmitted to a receiver external to the patient's body.

It is a yet further object of the invention to provide a Doppler blood flow monitoring device comprising:

a sensor cuff capable of being secured around an exterior surface of a vessel;

a first transducer attached to the sensor cuff for generating signals into and through the vessel;

a second transducer attached to the sensor cuff for receiving said signals; and

a transmitter in communication with the second transducer and capable of wirelessly transmitting a signal external to the patient's body.

It is a yet further object of the invention to provide a Doppler blood flow monitoring system comprising:

a sensor cuff capable of being secured around an exterior surface of a vessel;

a first transducer attached to the sensor coif for generating signals into and through the vessel;

a second transducer attached to the sensor cuff for receiving said signals;

a transmitter in communication with the second transducer and capable of wirelessly transmuting a signal external to the patient's body; and

a receiver for receiving said signal and being capable of emitting an audible signal or sound.

It is a yet further object of the invention to provide a pulsed Doppler blood flow monitoring device comprising:

a sensor cuff capable of being secured around an exterior surface of a patient's vessel;

a transducer containing both a transmitter and a receiver attached to the sensor cuff for generating signals into and through the vessel; and

a transmitter in communication with the transducer and capable of wirelessly transmitting a signal external to the patient's body.

It is a yet further object of the invention to provide a pulsed Doppler blood flow monitoring device comprising:

a sensor cuff capable of being secured around an exterior surface of a patient's vessel;

a transducer containing both a transmitter and a receiver attached to the sensor cuff for generating signals into and through the vessel;

a transmitter in communication with the transducer and capable of wirelessly transmitting a signal external to the patient's body; and

a receiver for receiving said signal and being capable of emitting an audible signal or sound.

These and other objects of the invention may be more easily understood from the description below.

### SUMMARY OF THE INVENTION

According to the invention, an implantable Doppler blood flow monitor detects blood flow in a vessel using a sensor cuff affixed to the external surface of the vessel. The implantable Doppler blood flow monitoring system detects microvascular anastomotic failures in free tissue transfers by detecting frequency shift relative to a transmitted wave through flowing blood. Traditional implantable Doppler blood flow monitoring systems require constant in-house monitoring by medical personnel, and wires most be passed through a subject's skin, which increases the risk of infection. According to the invention, a totally implantable wireless Doppler blood flow monitor is not tethered, and it allows for remote monitoring of free tissue transfers and other blood flow monitoring which is part of any patient diagnosis or treatment.

There are two basic variations of Doppler flow monitoring: pulsed wave Doppler (PWD) and continuous wave Doppler (CWD). These two technologies functionally differ in that the PWD is capable of resolving blood flow at desired depths with an upper limit on the blood flow velocity, while the CWD cannot resolve blood flow at a user-defined depth. However, there is no an upper limit on the blood flow velocity for CWDs. Also, the power requirements for a pulsed wave Doppler can be prohibitive in some cases.

According to an embodiment of the invention, a Doppler blood flow monitoring device comprises a sensor cuff, at least first and second transducers, and a transmitter. The sensor cuff is capable of being secured around the exterior surface of a vessel. A first transducer attached to the sensor cuff generates signals into and through medium in the vessel, and a second transducer attached to the sensor cuff receives the signals from the first transducer. The transmitter, which is in electrical communication with at least the second transducer, is capable of transmitting signals to a receiver external to the patient's body. The receiver is capable of generating signals such as audible signals or sounds that indicate flow in the vessel. The transmitter, a battery, and appropriate circuitry are contained in a transmitter module.

According to another embodiment of the invention, a method for monitoring blood flow in a vessel in a patient's body using Doppler technology comprises affixing a sensor cuff containing at least two transducers or sensors to the external surface of the vessel, causing signals to be transmitted from one transducer through medium in the vessel to a second transducer, transmitting signals from the second transducer to a transmitter, and transmitting a signal or signals to a receiver outside the patient's body, whereupon an audible signal or sound is generated commensurate with flow.

According to another embodiment of the invention, a Doppler blood flow monitoring device comprises a sensor cuff, a transducer and a transmitter. The sensor cuff is capable of being secured around the exterior surface of a vessel. The transducer attached to the sensor cuff generates signals into and through medium in the vessel and then receives reflected signals. The transmitter, which is in electrical communication with the transducer, is capable of transmitting RF signals to a receiver external to the patient's body. The receiver is capable of generating signals such as audible signals or sounds that indicate flow in the vessel. The transmitter, a battery, and appropriate circuitry ate contained in a transmitter module.

According to another embodiment of the invention, the transducers transmit and receive ultrasound signals or waves. However, it is within the scope of the invention that other wave technology, such as infrared signals or waves, may be used instead. For example, a first transducer may be a light-emitting diode to transmit an IR signal, and a second transducer may be a photo transistor to receive that signal.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It Is to be expressly understood, however, that the drawings are for the purpose of illustration and description only and are not intended as a definition of the limits of the invention.

### BRIEF DESCRIPTION OF THE DRAWINGS

FIG. 1 is a representation of a Doppler blood flow monitoring system according to the invention;

FIGS. 2A and 2B are each a schematic representation of a cross-sectional view of a sensor cuff useful according to the invention;

FIG. 3 is a block diagram of electronic components of a Doppler blood flow monitoring system according to the invention;

FIG. 4 is a block diagram of components of a transmitter module according to the invention; and

FIG. 5 is a representation of a receiver useful according to the invention.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right upper, lower, front, back, and derivatives thereof, relate to the orientation of the elements shown in the drawings and are not limiting upon the claims unless expressly recited therein.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality).

A Doppler blood flow monitoring system according to the invention comprises a sensor cuff, at least one transducer attached to the sensor cuff, a transmitter, and a receiver. The device also includes a first transducer or probe element and a second transducer or probe element that are coupled to and held by the sensor cuff or collar, wherein the first transducer is facing the second transducer such that a CW Doppler signal may be transmitted by the first transducer through the blood vessel and be received by the second transducer. In another embodiment, there may be a single transducer whereby the transmitter and receiver transducers are a single unit. The device further includes electronics that are connected to the transducers via wires. The electronics generate a pulse wave or in a first embodiment a CW Doppler signal and interpret the received signal. The electronics have wireless communications capability to send information outside the body. The surgeon will surgically remove the electronics, the wires, and the transducers, leaving the sensor cuff in place to be resorbed.

The invention can perhaps be appreciated better by making reference to the drawings herein.

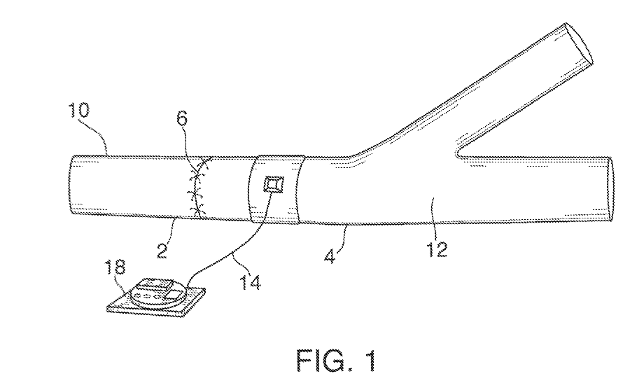

FIG. 1 represents a Doppler blood flow monitoring system according to the invention where vessels 2, 4 have been joined at vascular anastomosis site 6. A sensor cuff 10 positioned on the outer surface 12 of vessel 4 is electrically connected through wires 14 to a transmitter module 18. Transmitter module 18 comprises, among other things, a transmitter and battery, not shown here.

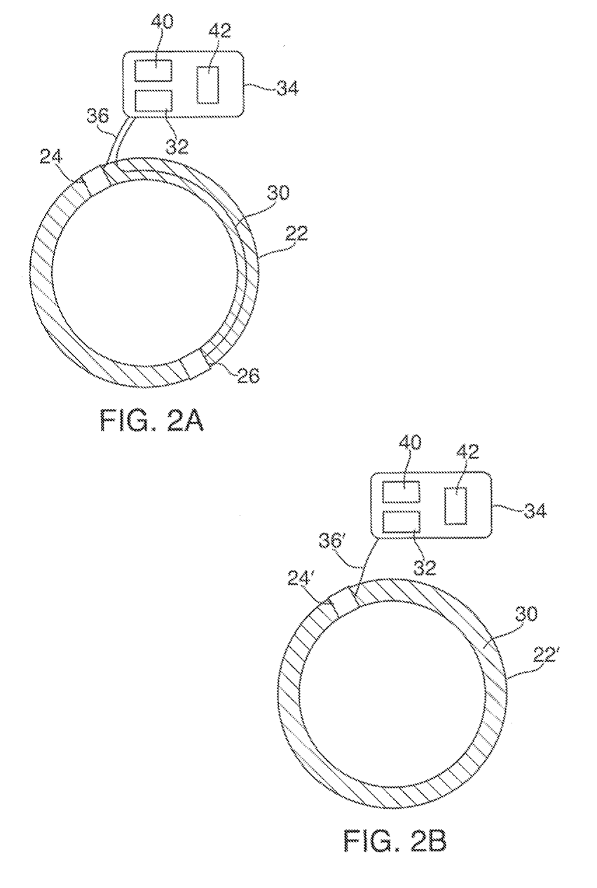

FIGS. 2A and 2B are each a schematic representation of a cross-section of a sensor cuff 22 or 22′ according to the invention. Sensor cuff 22 comprises a first transducer 24 for generating a signal through medium such as blood in a vessel. A second transducer 26 is positioned approximately 180° opposite transducer 24 to receive the signal, at a shift commensurate with Doppler shift theory. Transducer 26 is in electrical communication through wires 30 with a transmitter 32 in a transmitter module 34. First transducer 24 is in electrical connection through wires 36 with transmitter module 34. Transmitter module 34 also comprises a battery 40 and other appropriate circuitry 42. In FIG. 2B, sensor cuff 22′ comprises a single transducer 24′ that generates a signal through the medium and then receives the signal at a shift commensurate with Doppler shift theory. Transducer 24′ is in electrical connection with transmitter module 34 through wires 36′.

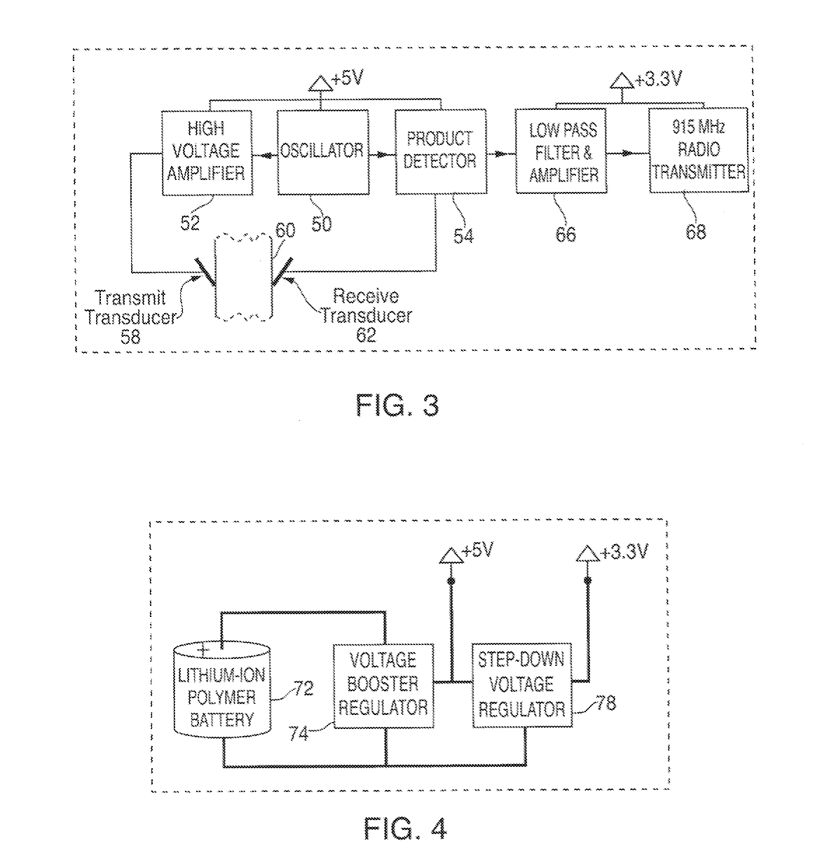

FIGS. 3 and 4 are each a block diagram that represents an important aspect of the invention. In FIG. 3, the electronics for the Doppler flow monitoring system comprise an oscillator 50 in communication with a high voltage amplifier 52 and a product or shift detector 54. Amplifier 52 is in communication with a transmitting or first transducer 58. A signal generated by transducer 58 through a blood vessel 60 is received by a receiving or second transducer 62. Transducer 62 communicates a signal to detector 54, which communicates a signal to a low pass filter and amplifier 66, which in turn communicates a signal to a radio transmitter 68.

FIG. 4 represents the power generation, or power management, of a transmitter module design of the system. A battery 72 is in communication with a voltager booster regulator 74 and a step-down voltage regulator 78.

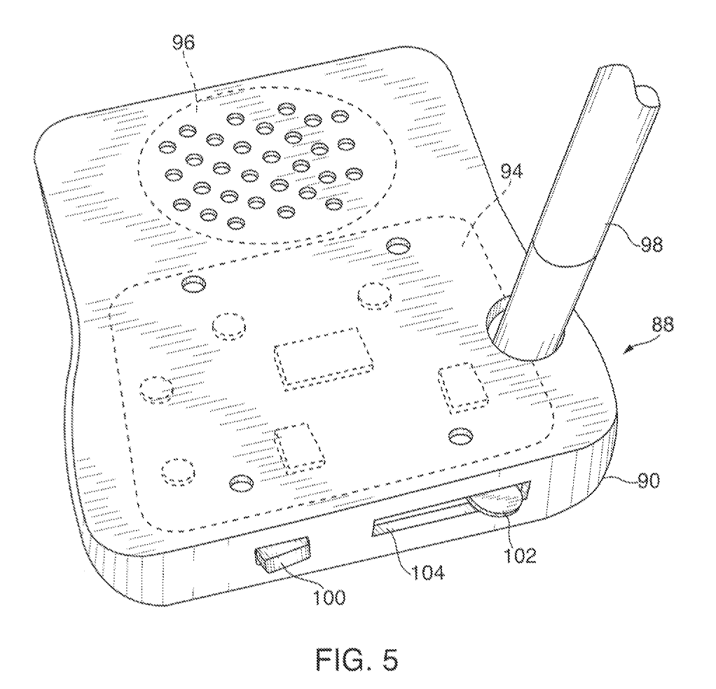

FIG. 5 is a representation of a receiver 88 useful according to the invention. Here, receiver 88 has a clear or other polymeric (e.g., polycarbonate or a similar rigid thermoplastic polymer or copolymer) case 90 wherein a circuit board 94 comprises components necessary to receive a signal from a transmitter module and to generate an audible signal or sound through a speaker 96. An antenna 98 receives the signal. An on/off switch 100 activates receiver 88, and a knob 102 in an opening 104 moves to adjust volume of the audible signal or sound.

The sensor cuff may comprise any suitable, physiologically acceptable material that is capable of being positioned or wrapped around or attached to a vessel. Such materials include any of many known polymeric materials, such as flexible polypropylene, polyethylene, polyurethane polymers or copolymers. The sensor cuff material may be positioned on a vessel prior to anastomosis, or it may be positioned after anastomosis where the cuff material segments will be affixed together. In a preferred embodiment, the sensor cuff material is resorbable, such as a magnesium-based polymeric material.

In one embodiment, the sensor cuff may comprise magnesium, possibly alloyed, with a single transducer or two transducers attached facing each other, which can result in a minimal diameter compared to the prior art. Magnesium will be resorbed into the body, leaving only a simple removal of the transmitter module with attached wires.

In the continuous wave Doppler embodiment of the invention, the sensor cuff will have at least two, preferably only two, transducers positioned on opposite surfaces on a sensor cuff, that is, approximately 180° apart. In the pulse wave Doppler embodiment of the invention, a single transducer may he used. The transducers are selected from known transducers capable of transmitting and receiving ultrasound and IR waves. Such transducers include, but are not limited to, lead zirconium titinate (PZT) crystal. Optionally the PZT probes can be replaced with a light emitting diode (LED) and a photo transistor, much like the circuitry used for measuring blood oxygen saturation (SPO2) and a pulse monitor. This technology will provide an equivalent blood flow monitor which may operate using IR as a Doppler replacement or other phenomenon to provide a device equivalent to the Doppler flow monitor.

In embodiments of the invention where IR waves are used for blood flow monitoring that may work on a Doppler or other physical principle, the primary benefits are a reduction in size and lower power requirements. This technology can also result in a longer implant life, permitting longitudinal biomarker data monitoring.

A transmitter is in electrical communication with at least the second transducer. Preferably such communication comprises, for example, copper or copper alloy wires of a diameter of from about 0.5 to about 1.0 mil. The transmitter comprises circuitry capable of receiving signals from the second transducer and transmitting an RF signal to a receiver external to the patient.

The transmitter module comprises a battery and a switching mechanism that is in electrical communication with the first transducer. The battery can be any physiologically acceptable low power battery suitable tor such operation.

The transmitter module must be physiologically acceptable and capable of protecting its components from bodily fluids. Preferably the transmitter module components are encapsulated and/or sealed in a hardenable polymeric material, such as a silicone or polysilicone material. One example of a useful material is NuSil MED12-6550, a polysilicone available from NuSil Technology LLC, Carpinteria, Calif. Other silicone elastomers are available from Nusil Technology as well.

### EXAMPLE

An implantable CWD flow monitor according to the invention (a preferred embodiment) was prepared from commercially available off-the-shelf parts. The resulting transmitter module had a diameter about the same as the diameter of a U.S. half dollar and less than about 0.32 inches thick without a battery and less than about 0.51 inches thick with a battery. This is smaller than modern pacemakers and implantable cardiac defibrillators.

The CWD was battery powered with a lithium-ion polymer battery with a nominal 3.7V and outfitted with a magnetically-actuated switching mechanism to extend the implant's useful lifetime. The switching mechanism allowed personnel to activate the device with a simple magnet near the implant site without disturbing the test animal. Several generations of the half dollar sized prototypes were developed. Each successive generation improved battery lifetime and incorporated more advanced electronics.

Development included an external RF receiver to provide an audio output of the transmitted RF signals from the implantable CWD. Particular attention was paid to the audio frequency ranges that could be expected when measuring both arterial and venous blood flow. This included the development of the necessary signal processing algorithms.

The sensing cuffs, with 5 MHz piezoelectric transducers, were custom designed and manufactured for the CWDs by Iowa Doppler Products, a commercial vendor specializing in Doppler sensors. Through the course of this work, several varieties of sensing cuffs were developed and considerable knowledge was exchanged with Iowa Doppler Products. The transmitter was a 915 MHz radio from Linx Technologies, and a Gilbert Cell Multiplier was used to extract Doppler frequency shifts. A printed circuit board comprised 1 oz. copper on FR-4 dielectric material.

The implantable CWDs were first tested in the laboratory using electrical/mechanical flow devices to ensure the proper operation during future in vivo experiments. A corn starch and water solution as well as a commercially available artificial blood surrogate were used as a part of the testing. Additionally, the devices had to be encapsulated in a biocompatible material. The encapsulation served as a moisture barrier; it was imperative that encapsulation would not hinder the functionality of the electronics nor be harmful or toxic to the animal. Several iterations of encapsulation tests were conducted before arriving on a solution that could be easily removed to service electronics after retrieval from an animal trial.

A protocol for an Internal Review Board (IRB) was prepared and received approval for in vivo testing in animals. Swine were chosen for the animal trials. Both arterial and venous blood flow were successfully measured with the CWD flow meter. (Due to the physiology of swine vessels, future trials are likely to be performed using more appropriate animals such as rabbits.)

Miniaturization is now possible based on the circuit and signal processing that has been developed and tested with the first implantable CWD prototypes. Size is proportional to battery power, and often the battery will determine the size of the implant. Miniaturization of an implantable device to the smallest extent possible holds a number of important prospects. Smaller devices equate to longer implant lifetimes due to decreased power requirements. A smaller device can be applied in many more locations on the body. Furthermore, smaller devices cause less distress for a patient, and the devices are less likely to be damaged by traumatic impacts, such as labs, due to less protrusion.

It is believed that the size of the transmitter module can be reduced to about half the current size, using commercial off-the-shelf parts. It is within the scope of the invention that the transmitter module could be reduced to a single silicon chip. An advantage of such a chip is that it could be small enough that the chip would not be noticed by a human subject after implantation.

One goal of the invention described herein to increase the salvage rate of free tissue following the occurrence of vascular thrombosis and preclude the need for lengthy hospital stays by making the monitoring device completely wireless. The risk for thrombosis is highest in the first few days after surgery. Even with advanced microsurgical techniques, failure rates between 4%-10% for free flaps and 15%-30% for replants have been reported. Also, it has been repotted that vascular graft failure rate is significantly higher, with less than 30% of grafts patent after three years. This indicates the need for chronic monitoring of patents over long periods. Salvage rates are greatly increased by the wireless capacity of the proposed solution, since information can be remotely transmitted to healthcare providers for frequent assessment, rather than periodic on-site patient exams.

The electronics shown above in the insert can be surgically removed rather easily, but the removal of the sensor cuff can be problematic. Thus, the desire to have a resorbable sensor cuff, where the wires can be simply be pulled out.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

We claim:

1. A continuous wave Doppler blood flow monitoring device comprising:
a sensor cuff capable of being secured around an exterior surface of a patient's vessel;
a first transducer attached to the sensor cuff for generating signals into and through the vessel;
a second transducer attached to the sensor cuff for receiving said signals; and
a transmitter in communication with the second transducer and capable of wirelessly transmitting a signal external to the patient's body.

2. The device of claim 1, wherein the sensor cuff is structured to be wrapped around a patient's vessel.

3. The device of claim 1, wherein the sensor cuff is comprised of material that is capable of being resorbed into the patient's body.

4. The device of claims 1, wherein each of the first transducer and the second transducer is removeably coupled to and supported by the sensor cuff.

5. The device of claim 1, wherein the first transducer and the second transducer face each other.

6. The device of claim 5, wherein a continuous wave Doppler signal can be transmitted by the first transducer through the vessel to be received by the second transducer.

7. Die device of claim 1, wherein the transmitter is in a transmitter module, and each of the first and second transducers are in electrical communication with the transmitter module.

8. The device of claim 7, wherein the transmitter module generates a continuous wave Doppler signal.

9. The device of claim 1, wherein the first transducer is a light-emitting diode and the second transducer is a photo transistor, such that an IR signal may be transmitted by the light-emitting diode through the vessel and be received by the photo transistor.

10. The device of claim 1, wherein the transmitter transmits an RF signal.

11. A system for monitoring blood flow, which comprises:
a continuous wave Doppler blood flow monitoring device of claim 1; and
a receiver to receive an RF signal from the transmitter and being capable of generating an audible sound or signal commensurate with detected flow.

12. A pulsed Doppler blood flow monitoring device comprising:
a sensor cuff capable of being secured around an exterior surface of a patient's vessel;
a transducer containing both a transmitter and a receiver attached to the sensor cuff for generating signals into and through the vessel; and
a transmitter in communication with the transducer and capable of wirelessly transmitting a signal external to the patient's body.

13. The device of claim 12, wherein the sensor cuff is structured to be wrapped around a patient's vessel.

14. The device of claim 12, wherein the sensor cuff is comprised of material that is capable of being resorbed into the patient's body.

15. The device of claim 12, wherein the transducer is removeably coupled to and supported by the sensor cuff.

16. The device of claim 12, wherein the transducer contains both a transmitter and receiver element.

17. The device of claim 16, wherein a pulse Doppler signal can be transmitted by the transducer through the vessel to be reflected to the transducer.

18. The device of claim 12, wherein the transmitter is in a transmitter module, and the transducer is in electrical communication with the transmitter module.

19. The device of claim 18, wherein the transmitter module generates a pulse wave Doppler signal.

20. The device of claim 12, wherein the transducer comprises a light-emitting diode and a photo transistor, such that an IR signal may be transmitted by the light-emitting diode through the vessel and be received by the photo transistor.

21. The device of claim 12, wherein the transmitter transmits an RF signal.

22. A system for monitoring blood flow, which comprises:
a pulsed Doppler blow flow monitoring device of claim 12; and
a receiver to receive an RF signal from the transmitter and being capable of generating an audible sound or signal commensurate with detected flow.
